(19) United States
(12) Patent Application Publication
(10) Pub. No.: US 2026/0355485 A1
(43) Pub. Date: Nov. 12, 2026

(54) Lighted Tattoo Machine

(71) Applicant: Bishop Tattoo Supply, Inc., Orange, CA (US)

(72) Inventors: Billy Chen, Orange, CA (US); Franco Vescovi, Lake Forest, CA (US)

(21) Appl. No.: 19/634,915

(22) Filed: Mar. 31, 2026

Related U.S. Application Data
(60) Provisional application No. 63/801,575, filed on May 7, 2025.

(51) Int. Cl. A61M 37/00

(52) U.S. Cl. CPC A61M 37/0076; A61M 2205/106; A61M 2205/502; A61M 2205/587

(57) ABSTRACT

A lighted tattoo machine, comprising: a power supply, which may be a removeable battery/controller; a tattoo machine that has an LED ring or LED lights; and a removeable grip that has a transparent or translucent portion at the needle end of the tattoo machine that is substantially aligned with the LED ring and allows light from the LED ring to illuminate a work area.

### CROSS-REFERENCE PARAGRAPH

This Application is a U.S. Non-Provisional Patent Application that claims the benefit of and priority to U.S. Utility Provisional Patent Application No. 63/801,575, which was filed on May 7, 2025, entitled LIGHTED TATTOO MACHINE, the contents of which are incorporated herein by this reference as though set forth in their entirety, and to which priority is claimed.

### FIELD OF DISCLOSURE

The present disclosure relates to tattoo machines that illuminate the subject being tattooed. More specifically, the present disclosure relates to a lighted tattoo machine that has a removeable battery power supply and controller, as well as a removeable grip that allows the light to illuminate the subject being tattooed.

### BACKGROUND

Tattooing is an art that requires precision and dexterity. The artist requires a well-lit surface and typically uses articulated and bright lamps. Some solutions have been integrated into tattoo machines including in U.S. Published Patent Application No. US20080089067A1, which obtains power from the same power supply as the tattoo motor and attaches to the outer surface of the tattoo machine. The light source is at the end of articulating arms. This Patent Application fails to disclose a battery powered and controlled tattoo machine that has lights integrated into the machine itself.

U.S. Patent No. 10226611 discloses a tattoo grip that integrates light provided by a fiberoptic cable. Specifically, the ‘611 Patent disclosures: “A grip light for use with a tattoo machine having an exterior grip surface extending from an upper grip portion downward toward an opposite working end and surrounding a channel extending through the upper portion to the working end, light transmissive material running through the grip from a light receiving surface to a bottom surface of the working end, and a light pipe inlet for receiving light from a light pipe into the light receiving surface and thereby permitting light from the light pipe to be projected out from working end of the grip light to illuminate an area therebelow.” The ‘611 Patent fails to disclose a battery powered and controlled tattoo machine that has lights integrated into the machine itself.

U.S. Patent No. 7393114 discloses a lighted grip and alligator clip cord for tattoo machine. The light is integrated into the grip, and the light power supply is connected to the lights via external wiring. The ‘114 Patent fails to disclose a battery powered and controlled tattoo machine that has lights integrated into the machine itself.

The current solutions for lighted tattoo machines are deficient because the light source is not integrated into the tattoo machine itself, the power source is not a battery, and/or the wires powering the light source are external to the tattoo machine. Thus, what is needed is a battery controlled and powered tattoo machine that has integrated lights and a removeable grip.

### SUMMARY

The following presents a simplified overview of the example embodiments in order to provide a basic understanding of some embodiments of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented hereinbelow. It is to be understood that both the following general description and the following detailed description are exemplary and explanatory only and are not restrictive.

In accordance with the embodiments disclosed herein, the present disclosure is directed to a tattoo machine with a removeable grip, an LED ring or other illumination component integrated within the tattoo motor portion, and that is powered by a removeable battery. The lights may be affixed to the spline of the machine and connects to the backplate of the machine by, as an example, a 2-pin molex connector, or a similar mate and lock type connector. The LED light ring may then connect to a 3-pin connector on the backplate, which might then be powered by the battery pack (or other power source) by mating the 3-pins with the contact plate on the battery. The battery, which may be the controller, may have a button or other mechanism that can control the one, two, or many more types of light provided by the LEDs (warm/cool/bright/soft/ultra bright/etc.) in variable intensity settings. The LED ring may have between one and several hundred light emitting diodes (LEDs) but may typically have between 3 and 20 LEDs formed into a ring shape. Preferably, the grip has a clear portion, such as an acrylic lens or light bar, which passes, directs, and/or diffuses the light to shine on the targeted areas of use. The grip may be permanent/durable or disposable.

One embodiment may be a lighted tattoo machine, comprising: a power supply; a tattoo machine portion; a removeable grip; wherein the tattoo machine portion comprises a motor, a plunger bar, and one or more LED lights; wherein the motor is configured to drive the plunger bar in a reciprocating manner, such that when at least one tattoo needle is operatively connected to the plunger bar, the at least one tattoo needle moves in a reciprocating manner; wherein the tattoo machine portion is operatively connected to the power supply, such that the power supply provides power to the motor and the one or more LED lights. The removeable grip may include an annular ring-shaped space that is configured to allow light from the one or more LED lights to pass through the removeable grip, such that a subject being tattooed is illuminated. The removeable grip may have a lens, wherein the lens covers the opening in the front of the removeable grip and is configured to allow light from the one or more LED lights to pass. The lens may be made of clear acrylic. The lens may be translucent. The lens may be transparent. The power supply may comprise a battery and may be removeably connectable to the tattoo machine portion. The power supply may further comprise a display screen and one or more buttons. The display screen or the one or more buttons may allow the user to do one or more of: turn the lighted tattoo machine on/off; increase/decrease a speed of the reciprocating plunger bar; adjust a stroke length of the reciprocating plunger bar; turn the one or more LED lights on/off; dim/brighten the one or more LED lights; and/or change the color of the one or more LED lights. The grip may be disposable. The grip may comprise one or more grip notches. An outer housing of the tattoo machine portion, grip, and/or power supply may be a material selected from the group of materials comprising one or more of: aluminum; metal; composites; carbon fiber; plastic; and rubber. An outer housing of the grip comprises a material selected from the group of materials consisting of: aluminum; metal; composites; carbon fiber; plastic; and rubber.

Another embodiment may be a lighted tattoo machine, comprising: a power supply; a tattoo machine portion; a grip; wherein the tattoo machine portion comprises a motor, a plunger bar, and a light source; wherein the motor is configured to drive the plunger bar in a reciprocating manner, such that when at least one tattoo needle is operatively connected to the plunger bar, the at least one tattoo needle moves in a reciprocating manner; wherein the tattoo machine portion is operatively connected to the power supply, such that the power supply provides power to the motor and the light source; wherein the grip is configured to allow light from the light source to pass through the grip, such that a subject being tattooed is illuminated. The light source is preferably one or more LED lights. The grip may comprise a lens, wherein the lens is configured to allow light from the one or more LED lights to pass. The LED lights may be controllable and configured to be turned on/off and/or dimmed/brightened.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an illustration of a front perspective view of one embodiment of the lighted tattoo machine.

FIG. 2 is an illustration of a back perspective view of one embodiment of the lighted tattoo machine.

FIG. 3 is an illustration of a front perspective view of one embodiment of the lighted tattoo machine showing the light shining out of the grip.

FIG. 4 is an illustration of a front perspective view of one embodiment of the lighted tattoo machine showing the battery removed.

FIG. 5 is an illustration of a side view of one embodiment of the removeable grip of the lighted tattoo machine.

FIG. 6 is an illustration of a front view of one embodiment of the removeable grip of the lighted tattoo machine.

FIG. 7 is an illustration of a front perspective view of another embodiment of a grip of the lighted tattoo machine.

FIG. 8 is an illustration of a side view of one embodiment of the tattoo machine portion of the lighted tattoo machine showing grip and battery connectors.

FIG. 9 is an illustration of a front view of one embodiment of the tattoo machine portion of the lighted tattoo machine.

FIG. 10 is an illustration of a cross-sectional view of one embodiment of the lighted tattoo machine of the present disclosure.

### DETAILED DESCRIPTION OF THE ILLUSTRATIVE EMBODIMENTS

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers, or steps. “Exemplary” means “an example of” and is not intended to convey an indication of a preferred or ideal embodiment. “Such as” is not used in a restrictive sense, but for explanatory purposes.

Disclosed are components that may be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in disclosed methods. Thus, if there are a variety of additional steps that may be performed it is understood that each of these additional steps may be performed with any specific embodiment or combination of embodiments of the disclosed methods.

The present methods and systems may be understood more readily by reference to the following detailed description of preferred embodiments and the examples included therein and to the Figures and their previous and following description.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 0.001-10% from the indicated number or range of numbers.

As used herein, the term “light source” refers to any illumination source or device that generates or provides light. Light source expressly includes any natural, artificial, digital, incandescent, luminescent, bioluminescent, chemical luminescent, halogen, gas discharge, light emitting diode, and/or combustion, source of light. Preferably, the light source of the present disclosure is light emitting diode (LED) lights. LED lights do not require high voltage or energy requirements, and they produce lower heat, which is very desirable. This makes LED lights an efficient light source, especially when the power supply is a battery.

Various embodiments are now described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

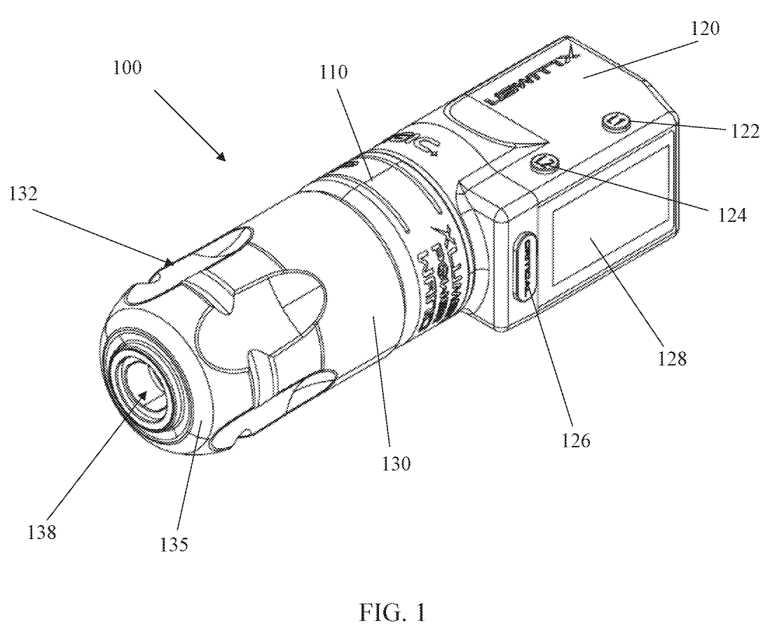

FIG. 1 is an illustration of a front perspective view of one embodiment of the lighted tattoo machine. As shown, in FIG. 1, lighted tattoo machine 100 may comprise grip 130, tattoo machine portion 110, and power supply 120. Preferably, the grip 130, tattoo machine portion 110, and power supply 120 may be made of metal, such as aluminum, plastic, rubber, and/or composite materials, which are easy to grip and hold securely. Power supply 120 is shown as a battery that also serves as the controller for tattoo machine portion 110. In other embodiments, the power supply may not be a battery, such as an inverter power supply that is plugged into a wall. FIG. 1 shows that power supply 120 may include a display screen 128, which may be LCD, LED, or any digital or analog display. Display screen 128 may be a touch screen with soft key functionality. Power supply 120 may include an on/off button 126, and one or more voltage up and down buttons 122, 124. Power supply 120 is preferably removeable and reconnectable. In other embodiments the power supply may be permanently connected to the tattoo machine portion. Display screen 128 and buttons 122, 124, 126, preferably allow the user to increase/decrease voltage, turn on/off the machine, turn on/off the light, adjust the light, and/or adjust the stroke length of the needle. Display screen 128 may be used to provide voltage and battery life information to the user.

Preferably grip 130 has grip notches 132 that allow for a comfortable and sure hold, lens 135, and needle opening 138. Lens 135 may preferably be a clear, translucent, or transparent, such that light generated by tattoo machine portion 110 is able to pass out of grip 130 and illuminate the subject or area that is being tattooed. Lens 135 may preferably be made of a durable plastic, such as acrylic, but it may be crystal or glass. In some embodiments, there is no lens and the light just passes through an opening in the grip where lens 135 is shown. In another embodiment, the lens may be the end of a fiberoptic cable.

In some embodiments, grip 130 may be removeable and disposable. Grip 130 may preferably connect to the front of the tattoo machine portion via threads/screwing. In other embodiments, some other connector may be used, such as friction, ball detent, magnets, snap, and the like.

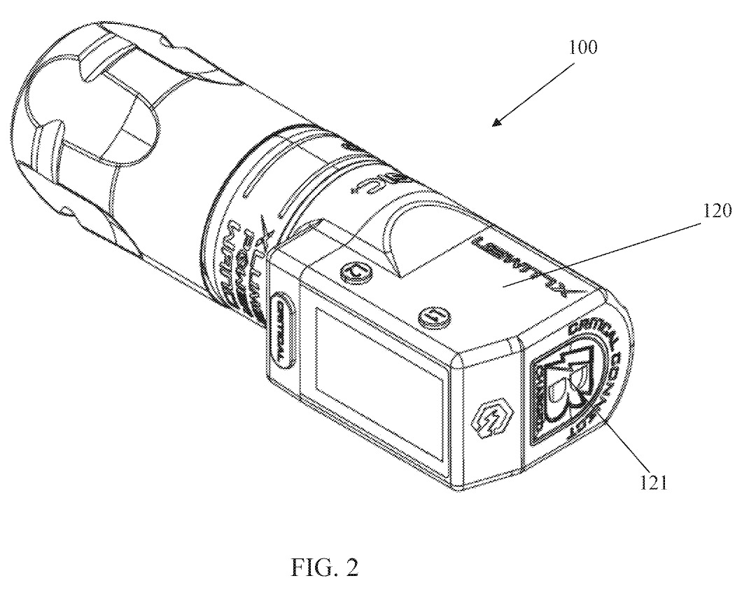

FIG. 2 is an illustration of a back perspective view of one embodiment of the lighted tattoo machine. As shown in FIG. 2, lighted tattoo machine 100 may have light control button 121 on power supply 120. In addition to turning the light on or off, light control button 121 may allow the user to increase or decrease the brightness, and/or cause flashing or strobe effects. light settings.

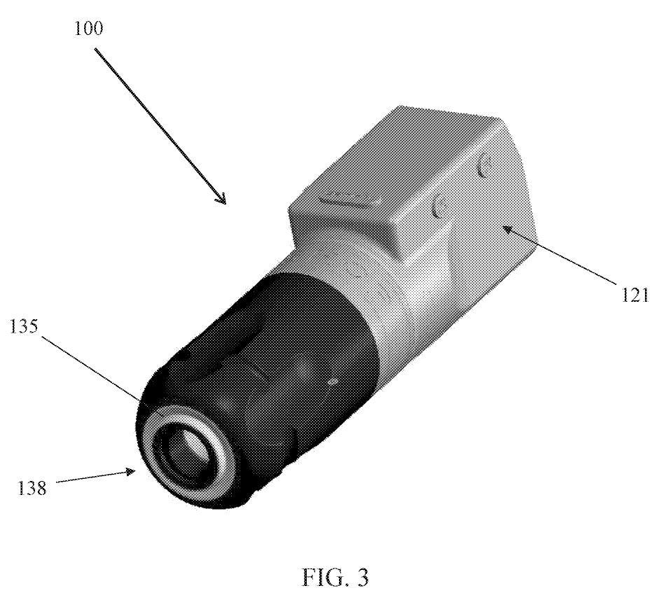

FIG. 3 is an illustration of a front perspective view of one embodiment of the lighted tattoo machine showing the light shining out of the grip. FIG. 3 shows that the light control button has been pressed so that light is passing through lens 135. In this manner the tattoo needle coming out of opening 138 would be surrounded by a ring of light.

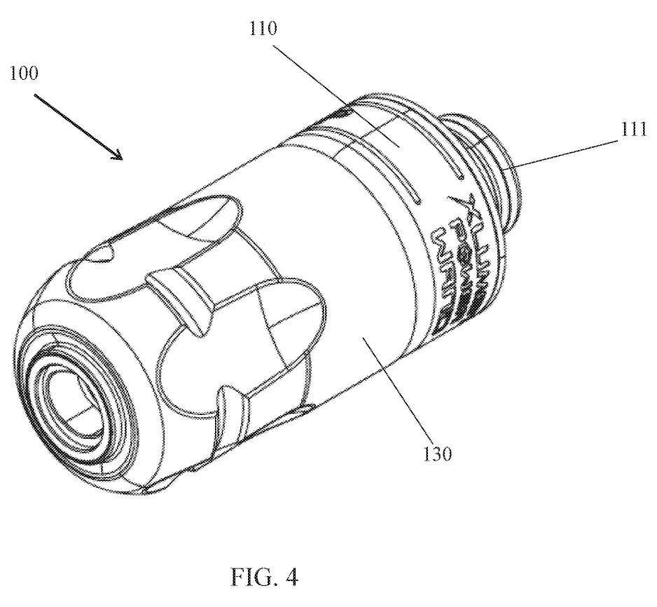

FIG. 4 is an illustration of a front perspective view of one embodiment of the lighted tattoo machine showing the battery removed. As shown in FIG. 4, the power supply may be disconnected from tattoo machine portion 110 of lighted tattoo machine 100. Power supply connector 111 may removeably connect, physically and electrically to the power supply.

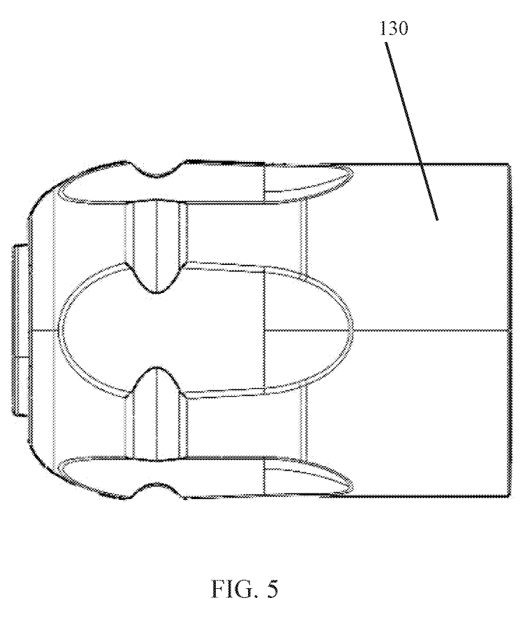

FIG. 5 is an illustration of a side view of one embodiment of the removeable grip of the lighted tattoo machine. As shown in FIG. 5, grip 130 has been disconnected from tattoo machine portion 110 of lighted tattoo machine 100.

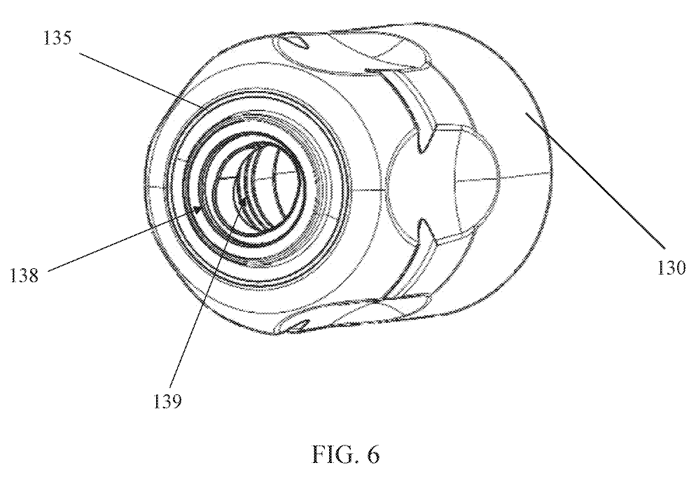

FIG. 6 is an illustration of a front view of one embodiment of the removeable grip of the lighted tattoo machine. As shown in FIG. 6, grip 130 has been disconnected from tattoo machine portion 110 of lighted tattoo machine 100. FIG. 6 also shows lens 135, tattoo needle opening 138 and connectors 139, which may be threaded connectors that matingly engage with threads of the tattoo machine portion so they can be screwed together.

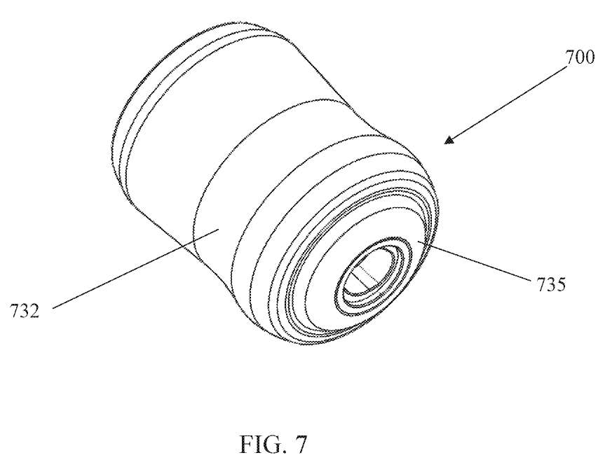

FIG. 7 is an illustration of a front perspective view of another embodiment of a grip of the lighted tattoo machine. FIG. 7 shows a disposable rubber grip 700, which may comprise grip portion 732 and lens 735. Similar to grip 130, grip 700 is removeably connectable to the tattoo machine portion, usually through threads. FIG. 7 shows how the grip may be a tacky material, such as rubber that does not require grip notches.

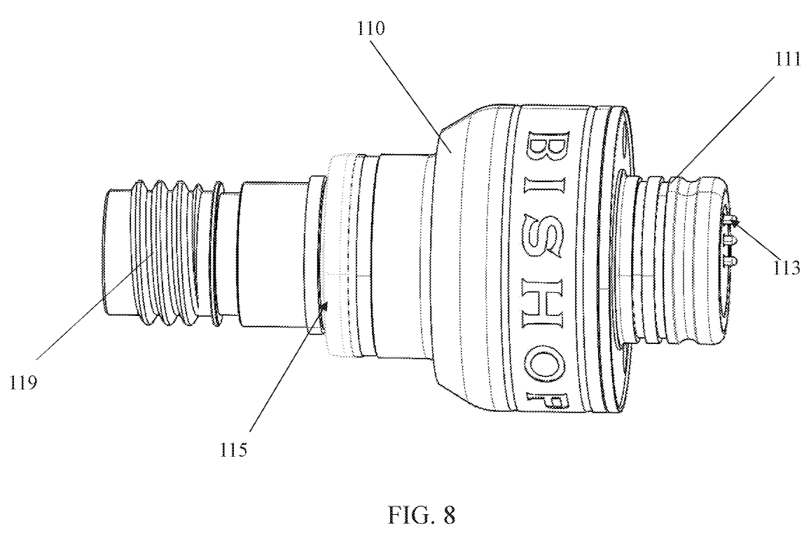

FIG. 8 is an illustration of a side view of one embodiment of the tattoo machine portion of the lighted tattoo machine showing grip and battery connectors. FIG. 8 shows tattoo machine portion 110 without a grip or power supply attached to it. Tattoo machine portion 110 may comprise LED lights 115, grip connection threads 119, and power supply connector 111, which may include 3-pin electrical connectors 113. Tattoo machine portion 110 may also have various connectors, screws, and O-rings to facilitate the connections with the power supply and grip and/or to enhance structural integrity.

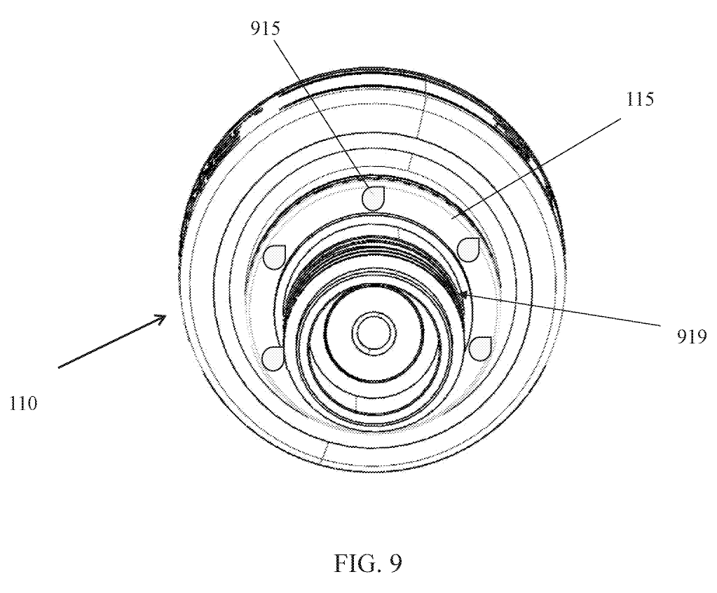

FIG. 9 is an illustration of a front view of one embodiment of the tattoo machine portion of the lighted tattoo machine. FIG. 9 shows that the LED lights 115 may be made out of a ring of LED lights 915 that are directed to shining forward towards the needle opening 919 of the tattoo machine portion 110.

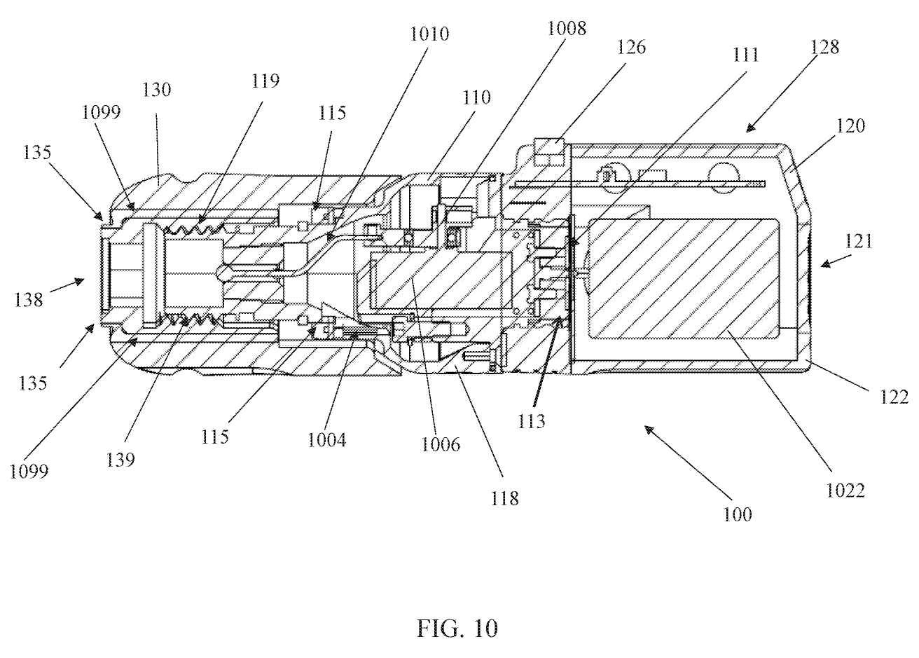

FIG. 10 is an illustration of a cross-sectional view of one embodiment of the lighted tattoo machine of the present disclosure. Lighted tattoo machine 100 may comprise tattoo machine portion 110, power supply 120, and grip 130. Power supply 120 may comprise housing 118, display screen 128, and various buttons, such as light control button 121 and power button 126. Power supply 120 also has a battery 1022. Although battery 1022 is preferably rechargeable without being removed from power supply 120, it may be removeable from power supply 120 and then recharged, or it may be removed from power supply 120 and replaced by a new or already recharged battery. As shown in FIG. 10, power supply 120 matingly engages with power supply connector 111 of tattoo machine portion 110, such that power supply 120 and tattoo machine portion 110 are physically connected and electrically connected, via 3-pin electrical connectors 113. In this manner, power supply 120 is securely, but removeable connected to tattoo machine portion. Wiring, such as light wiring harness 1004, electrically connects 3-pin electrical connectors 113 to the parts of tattoo machine portion 110 that need power.

Tattoo machine portion 110 may comprise: motor 1006, which may drive the reciprocating tattoo needles; housing 118; drive shaft 1008, which interacts with an offset cam to convert the rotational motion of drive shaft 1008 into linear motion, such that plunger bar 1010 is driven back and forth in a reciprocating manner. Plunger bar 1010 may be constrained by plunger bar brushing. In some embodiments, the tattoo machine portion may have a clutch that allows modification to the stroke length of the plunger bar, which allows the user to increase or decrease the depth of needle penetration into the subject.

As shown, light wiring harness 1004, comes out of the back of LED lights 115 and connects to the 2-pin molex connector to get power from power supply 120. When light control button 121 is pushed, LED lights 115 illuminate, turn off, or get brighter.

As shown in FIG. 10, grip 130 is removeably connected to tattoo machine portion 110 via threaded connectors 119, 139. When connected, an annular ring of space 1099 is aligned with LED lights 115, such that light generated by LED lights 115 passes through grip 130 and out of lens 135. In this manner, the light is able to illuminate what is being tattooed by the needle connected to tattoo machine portion 110 through opening 138. FIG. 10 shows that grip 130 substantially encases or covers a front of tattoo machine portion 110, such that LED lights 115 are protected by grip 130. Light from LED lights 115 passes through an open space or hole in grip 130, such as the annular ring of space 1099, shown in FIG. 10, which substantially runs the length of grip 130.

In other embodiments, the light may be transferred via a fiber optic cable. In one embodiment, the light may pass through a focusing lens that makes it brighter. The focusing lens may be moveable within the annular ring to further focus the light. In some embodiments, the Kelvins of the LED Lights may be adjusted to be warmer or cooler.

In various embodiments, the LED lights may be any shape or type of LED, including, but not limited to, Chip on Board, Surface Mounted Device, Dual In Line Package, Red Green Blue White combined diodes, Smart LED, Bicolor, and or Dimmable.

The previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow; plain meaning derived from grammatical organization or punctuation; the number or type of embodiments described in the specification.

It will be apparent to those of ordinary skill in the art that various modifications and variations may be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A lighted tattoo machine, comprising:
a power supply;
a tattoo machine portion;
a removeable grip;
wherein said tattoo machine portion comprises a motor, a plunger bar, and one or more LED lights;
wherein said motor is configured to drive said plunger bar in a reciprocating manner, such that when at least one tattoo needle is operatively connected to said plunger bar, said at least one tattoo needle moves in a reciprocating manner;
wherein said tattoo machine portion is operatively connected to said power supply, such that said power supply provides power to said motor and said one or more LED lights.

2. The lighted tattoo machine of claim 1, wherein said removeable grip comprises an annular ring-shaped space that is configured to allow light from said one or more LED lights to pass through said removeable grip, such that a subject being tattooed is illuminated.

3. The lighted tattoo machine of claim 2, wherein said removeable grip comprises a lens, wherein said lens covers an opening in a front of said removeable grip and is configured to allow light from said one or more LED lights to pass.

4. The lighted tattoo machine of claim 3, wherein said lens is made of clear acrylic.

5. The lighted tattoo machine of claim 3, wherein said lens is translucent.

6. The lighted tattoo machine of claim 3, wherein said lens is transparent.

7. The lighted tattoo machine of claim 3, wherein said power supply comprises a battery and is removeably connectable to said tattoo machine portion.

8. The lighted tattoo machine of claim 7, wherein said power supply further comprises a display screen and one or more buttons.

9. The lighted tattoo machine of claim 8, wherein said display screen or said one or more buttons allow a user to do one or more of: turn said lighted tattoo machine on/off; increase/decrease a speed of said plunger bar; adjust a stroke length of said plunger bar; turn said one or more LED lights on/off; dim/brighten said one or more LED lights; and change the color of said one or more LED lights.

10. The lighted tattoo machine of claim 8, wherein said display screen or said one or more buttons are configured to turn on and off said one or more LED Lights.

11. The lighted tattoo machine of claim 8, wherein said display screen or said one or more buttons are configured to dim and brighten said one or more LED Lights.

12. The lighted tattoo machine of claim 8, wherein said display screen or said one or more buttons are configured to change the color of said one or more LED Lights.

13. The lighted tattoo machine of claim 3, wherein said removeable grip is disposable.

14. The lighted tattoo machine of claim 3, wherein said removeable grip comprises one or more grip notches.

15. The lighted tattoo machine of claim 3, wherein an outer housing of said tattoo machine portion comprises a material selected from the group of materials consisting of: aluminum; metal; composites; carbon fiber; plastic; and rubber.

16. The lighted tattoo machine of claim 3, wherein an outer housing of said removeable grip comprises a material selected from the group of materials consisting of: aluminum; metal; composites; carbon fiber; plastic; and rubber.

17. The lighted tattoo machine of claim 3, wherein an outer housing of said power supply comprises a material selected from the group of materials consisting of: aluminum; metal; composites; carbon fiber; plastic; and rubber.

18. A lighted tattoo machine, comprising:
a power supply;
a tattoo machine portion;
a grip;
wherein said tattoo machine portion comprises a motor, a plunger bar, and a light source;
wherein said motor is configured to drive said plunger bar in a reciprocating manner, such that when at least one tattoo needle is operatively connected to said plunger bar, said at least one tattoo needle moves in a reciprocating manner;
wherein said tattoo machine portion is operatively connected to said power supply, such that said power supply provides power to said motor and said light source;
wherein said grip is configured to allow light from said light source to pass through said grip, such that a subject being tattooed is illuminated.

19. The lighted tattoo machine of claim 18, wherein said light source is one or more LED lights;
wherein said grip comprises a lens;
wherein said lens is configured to allow light from said one or more LED lights to pass.

20. The lighted tattoo machine of claim 19, wherein said LED lights are controllable and configured to be turned on/off and dimmed/brightened.
